(19) United States
(12) Patent Application Publication
(10) Pub. No.: US 2020/0219228 A1
(43) Pub. Date: Jul. 9, 2020

(54) REAL-TIME REGIONAL ENHANCEMENT IMAGING AND DISPLAY FOR ULTRASOUND IMAGING

(71) Applicant: Shenzhen Mindray Bio-Medical Electronics Co., Ltd., Shenzhen (CN)

(72) Inventors: Ting-lan Ji, Morgan Hill, CA (US); Donglai Liu, San Jose, CA (US); Glen W. McLaughlin, San Carlos, CA (US)

(21) Appl. No.: 16/242,664

(22) Filed: Jan. 8, 2019

(51) Int. Cl. G06T 3/40; A61B 8/00; A61B 8/08

(52) U.S. Cl. CPC G06T 3/40; A61B 8/463; A61B 8/483; A61B 8/481; A61B 8/486

(57) ABSTRACT

Systems and methods for performing ultrasound imaging. Main channel domain data for performing ultrasound imaging of a subject area for performing ultrasound imaging of the subject area is collected. Further, region of interest (“ROI”) channel domain data for performing ultrasound imaging of a ROI within the subject area is collected. One or more ultrasound images of the subject area can be formed using the main channel domain data. Additionally, one or more ROI ultrasound images of the ROI within the subject can be formed independently from the one or more main ultrasound images of the subject area using the ROI channel domain data of the ROI within the subject area. Subsequently, the one or more ROI ultrasound images can be displayed concurrently with the one or more main ultrasound images.

### TECHNICAL FIELD

This disclosure relates to ultrasound imaging. Specifically, this disclosure relates to forming main ultrasound images of a subject area and separately forming ROI ultrasound images of a ROI within the subject area for concurrent display with the main ultrasound images.

### BACKGROUND OF THE INVENTION

Ultrasound imaging is widely used for examining a wide range of materials and objects across a wide array of different applications. Ultrasound imaging provides a fast and easy tool for analyzing materials and objects in a non-invasive manner. As a result, ultrasound imaging is especially common in the practice of medicine as an ailment diagnosis, treatment, and prevention tool. Specifically, because of its relatively non-invasive nature, low cost and fast response time ultrasound imaging is widely used throughout the medical industry to diagnose and prevent ailments. Further, as ultrasound imaging is based on non-ionizing radiation it does not carry the same risks as other diagnosis imaging tools, such as X-ray imaging or other types of imaging systems that use ionizing radiation.

In many ultrasound applications, specific areas of interest within an ultrasound image of a subject area are more important than other areas in the subject area. For example, when imaging a tumor, the tumor itself is of greater interest to a doctor than areas separate from the tumor. Accordingly, it is beneficial to provide an operator with image enhancement capabilities, e.g. improved image resolution, of a ROI of a subject area in an ultrasound image.

Acoustic zoom, otherwise known as front-end zoom, has been used to amplify a portion of an ultrasound image within a user-specified ROI so that a user can better visualize details of the portion of the image that includes the ROI. Specifically, since the selected ROI box is typically smaller than the full size image, which usually means less transmit firings with less number of receive lines, a user can obtain higher frame rate images, e.g. improved temporal resolution, of the ROI. However acoustic zoom techniques for enhanced imaging in a ROI suffer from deficiencies. Specifically, through acoustic zoom techniques, only a zoomed image portion is displayed. Therefore, the relationship between the zoomed image portion and the rest of the entire image is lost at a detriment to ultrasound operators.

Alternately, an ultrasound image can be magnified by a linear interpolation to enhance images within a ROI. This magnification is often called back-end zoom or display zoom. Magnifying an ultrasound image to create enhanced ultrasound images also suffers from deficiencies. Specifically, a zoomed image portion of an ultrasound image created using interpolation does not have enhanced image quality except for magnification.

There therefore, exist needs for systems and methods that facilitate enhanced ultrasound imaging beyond magnification enhancement while allowing for concurrent display of enhanced ultrasound images with main ultrasound images.

### SUMMARY

According to various embodiments, a method for performing ultrasound imaging includes collecting main channel domain data of a subject area for performing ultrasound imaging of the subject area. Further, ROI channel domain data for performing ultrasound imaging of a ROI within the subject area can be collected. One or more main ultrasound images of the subject area can be formed using the main channel domain data. Additionally, one or more ROI ultrasound images within the subject area can be independently formed from the one or more main ultrasound images of the subject area using the ROI channel domain data of the ROI within the subject area. The one or more ROI ultrasound images can be displayed concurrently with the one or more main ultrasound images.

In certain embodiments, a system for performing ultrasound imaging includes an ultrasound transducer and a main processing console. The ultrasound transducer can be configured to collect main channel domain data for performing ultrasound imaging of a subject area. Further, the ultrasound transducer can be configured to collect ROI channel domain data for performing ultrasound imaging of a ROI within the subject area. The main processing console can be configured to form one or more main ultrasound images of the subject area using the main channel domain data. Further, the main processing console can be configured to form one or more ROI ultrasound images of the ROI within the subject area independently from the one or more main ultrasound images of the subject area using the ROI channel domain data of the ROI within the subject area. The main processing console can also be configured to display the one or more ROI ultrasound images concurrently with the one or more main ultrasound images.

In various embodiments, a system for performing ultrasound imaging includes one or more processors and a computer-readable medium providing instructions accessible to the one or more processors to cause the one or more processors to perform operations including collecting main channel domain data for performing ultrasound imaging of a subject area. The instructions can further cause the one or more processors to collect ROI channel domain data for performing ultrasound imaging of a ROI within the subject area. Additionally, the instructions can cause the one or more processors to form one or more main ultrasound images of the subject area using the main channel domain data. The instructions can also cause the one or more processors to form one or more ROI ultrasound images of the ROI within the subject area independently from the one or more main ultrasound images of the subject area using the ROI channel domain data of the ROI within the subject area. Further, the instructions can cause the one or more processors to display the one or more ROI ultrasound images concurrently with the one or more main ultrasound images.

### BRIEF DESCRIPTION OF THE DRAWINGS

FIG. 1 illustrates an example of an ultrasound system.

FIG. 2 is a flowchart of an example method for enhancing ultrasound images of a ROI within a subject area and displaying the ROI ultrasound images concurrently with main ultrasound images of the subject area.

FIG. 3 shows an example imaging sequence of ROI ultrasound frames and main ultrasound frames collected according to varying data acquisition parameters.

FIG. 4 shows another example imaging sequence of ROI ultrasound frames and main ultrasound frames collected according to varying data acquisition parameters.

FIG. 5 shows yet another example imaging sequence collected according to varying acquisition parameters.

FIG. 6 shows an example ultrasound image display format, where ROI ultrasound images, e.g. enhanced ROI ultrasound images, are displayed within main ultrasound images.

FIG. 7 shows an example ultrasound image display format, where ROI ultrasound images, e.g. enhanced ROI ultrasound images, are displayed adjacent to main ultrasound images.

FIG. 8 shows another example flowchart of an example method for independently generating ROI ultrasound images of a subject area from main ultrasound images of the subject area for concurrent display of the ROI and main ultrasound images.

### DETAILED DESCRIPTION

According to various embodiments, a method for performing ultrasound imaging includes collecting main channel domain data of a subject area for performing ultrasound imaging of the subject area. Further, ROI channel domain data for performing ultrasound imaging of a ROI within the subject area can be collected. One or more main ultrasound images of the subject area can be formed using the main channel domain data. Additionally, one or more ROI ultrasound images within the subject area can be independently formed from the one or more main ultrasound images of the subject area using the ROI channel domain data of the ROI within the subject area. The one or more ROI ultrasound images can be displayed concurrently with the one or more main ultrasound images.

In certain embodiments, a system for performing ultrasound imaging includes an ultrasound transducer and a main processing console. The ultrasound transducer can be configured to collect main channel domain data for performing ultrasound imaging of a subject area. Further, the ultrasound transducer can be configured to collect ROI channel domain data for performing ultrasound imaging of a ROI within the subject area. The main processing console can be configured to form one or more main ultrasound images of the subject area using the main channel domain data. Further, the main processing console can be configured to form one or more ROI ultrasound images of the ROI within the subject area independently from the one or more main ultrasound images of the subject area using the ROI channel domain data of the ROI within the subject area. The main processing console can also be configured to display the one or more ROI ultrasound images concurrently with the one or more main ultrasound images.

In various embodiments, a system for performing ultrasound imaging includes one or more processors and a computer-readable medium providing instructions accessible to the one or more processors to cause the one or more processors to perform operations including collecting main channel domain data for performing ultrasound imaging of a subject area. The instructions can further cause the one or more processors to collect ROI channel domain data for performing ultrasound imaging of a ROI within the subject area. Additionally, the instructions can cause the one or more processors to form one or more main ultrasound images of the subject area using the main channel domain data. The instructions can also cause the one or more processors to form one or more ROI ultrasound images of the ROI within the subject area independently from the one or more main ultrasound images of the subject area using the ROI channel domain data of the ROI within the subject area. Further, the instructions can cause the one or more processors to display the one or more ROI ultrasound images concurrently with the one or more main ultrasound images.

Some of the infrastructure that can be used with embodiments disclosed herein is already available, such as general-purpose computers, computer programming tools and techniques, digital storage media, and communications networks. A computing device may include a processor such as a microprocessor, microcontroller, logic circuitry, or the like. The processor may include a special purpose processing device such as an ASIC, PAL, PLA, PLD, FPGA, or other customized or programmable device. The computing device may also include a computer-readable storage device such as non-volatile memory, static RAM, dynamic RAM, ROM, CD-ROM, disk, tape, magnetic, optical, flash memory, or other computer-readable storage medium.

Various aspects of certain embodiments may be implemented using hardware, software, firmware, or a combination thereof. As used herein, a software module or component may include any type of computer instruction or computer executable code located within or on a computer-readable storage medium. A software module may, for instance, comprise one or more physical or logical blocks of computer instructions, which may be organized as a routine, program, object, component, data structure, etc., that performs one or more tasks or implements particular abstract data types.

In certain embodiments, a particular software module may comprise disparate instructions stored in different locations of a computer-readable storage medium, which together implement the described functionality of the module. Indeed, a module may comprise a single instruction or many instructions, and may be distributed over several different code segments, among different programs, and across several computer-readable storage media. Some embodiments may be practiced in a distributed computing environment where tasks are performed by a remote processing device linked through a communications network.

The embodiments of the disclosure will be best understood by reference to the drawings. The components of the disclosed embodiments, as generally described and illustrated in the figures herein, could be arranged and designed in a wide variety of different configurations. Furthermore, the features, structures, and operations associated with one embodiment may be applicable to or combined with the features, structures, or operations described in conjunction with another embodiment. In other instances, well-known structures, materials, or operations are not shown or described in detail to avoid obscuring aspects of this disclosure.

Thus, the following detailed description of the embodiments of the systems and methods of the disclosure is not intended to limit the scope of the disclosure, as claimed, but is merely representative of possible embodiments. In addition, the steps of a method do not necessarily need to be executed in any specific order, or even sequentially, nor need the steps be executed only once.

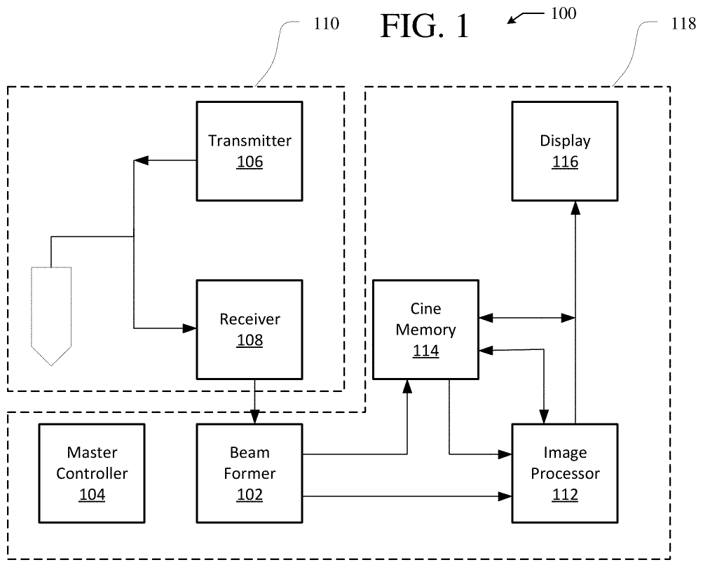

FIG. 1 illustrates an example of an ultrasound system 100. The ultrasound system 100 shown in FIG. 1 is merely an example system and in various embodiments, the ultrasound system 100 can have less components or additional components. The ultrasound system 100 can be an ultrasound system where the receive array focusing unit is referred to as a beam former 102, and image formation can be performed on a scanline-by-scanline basis. System control can be centered in the master controller 104, which accepts operator inputs through an operator interface and in turn controls the various subsystems. For each scan line, the transmitter 106 generates a radio-frequency (RF) excitation voltage pulse waveform and applies it with appropriate timing across the transmit aperture (defined by a sub-array of active elements) to generate a focused acoustic beam along the scan line. RF echoes received by the receive aperture 108 of the transducer 110 are amplified and filtered by the receiver 108, and then fed into the beam former 102, whose function is to perform dynamic receive focusing; i.e., to re-align the RF signals that originate from the same locations along various scan lines.

The image processor 112 can perform processing specific to active imaging mode(s) including 2D scan conversion that transforms the image data from an acoustic line grid to an X-Y pixel image for display. For Spectral Doppler mode, the image processor 112 can perform wall filtering followed by spectral analysis of Doppler-shifted signal samples using typically a sliding FFT-window. The image processor 112 can also generate the stereo audio signal output corresponding to forward and reverse flow signals. In cooperation with the master controller 104, the image processor 112 also can format images from two or more active imaging modes, including display annotation, graphics overlays and replay of cine loops and recorded timeline data.

The cine buffer 114 provides resident digital image storage for single image or multiple image loop review, and acts as a buffer for transfer of images to digital archival devices. On most systems, the video images at the end of the data processing path can be stored to the cine memory. In state-of-the-art systems, amplitude-detected, beamformed data may also be stored in cine memory 114. For spectral Doppler, wall-filtered, baseband Doppler 1/Q data for a user-selected range gate can be stored in cine memory 114. Subsequently, the display 116 can display ultrasound images created by the image processor 112 and/or images using data stored in the cine memory 114.

The beam former 102, the master controller 104, the image processor 112, the cine memory 114, and the display can be included as part of a main processing console 118 of the ultrasound system 100. In various embodiments, the main processing console 118 can include more or fewer components or subsystems. The ultrasound transducer 110 can be incorporated in an apparatus that is separate from the man processing console 118, e.g. in a separate apparatus that is wired or wirelessly connected to the main processing console 118. This allows for easier manipulation of the ultrasound transducer 110 when performing specific ultrasound procedures on a patient. Further, the transducer 110 can be an array transducer that includes an array of transmitting and receiving elements for transmitting and receiving ultrasound waves.

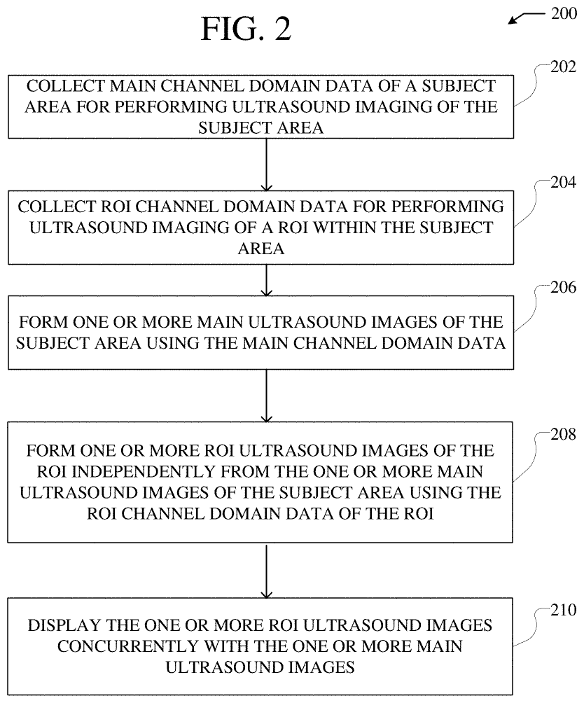

FIG. 2 is a flowchart 200 of an example method for enhancing ultrasound images of a ROI within a subject area and displaying the ROI ultrasound images concurrently with main ultrasound images of the subject area. The example method shown in FIG. 2, and other methods and techniques for ultrasound imaging described herein, can be performed by an applicable ultrasound imaging system, such as the ultrasound system 100 shown in FIG. 1. For example, the example methods and techniques for ultrasound imaging described herein can be implemented using either or both the ultrasound transducer 110 and the main processing console 118, e.g. the image processor 112, of the ultrasound system 100.

As discussed previously, in many ultrasound applications, specific areas of interest within an ultrasound image of a subject area are more important than other areas in the subject area. For example, when imaging a tumor, the tumor itself is of greater interest to a doctor than areas separate from the tumor. Accordingly, it is beneficial to provide an operator with image enhancement capabilities, e.g. improved image resolution, of a ROI of a subject area in an ultrasound image.

Acoustic zoom, otherwise known as front-end zoom, has been used to amplify a portion of an ultrasound image within a user-specified ROI so that a user can better visualize details of the portion of the image that includes the ROI. Specifically, since the selected ROI box is typically smaller than the full size image, which usually means less transmit firings with less number of receive lines, a user can obtain higher frame rate images, e.g. improved temporal resolution, of the ROI. However acoustic zoom techniques for enhancing imaging in a ROI suffer from deficiencies. Specifically, through acoustic zoom techniques, only a zoomed image portion is displayed. Therefore, the relationship between the zoomed image portion and the rest of the entire image is lost at a detriment to ultrasound operators.

Alternately, an ultrasound image can be magnified by a linear interpolation to enhance images within a ROI. This magnification is often called back-end zoom or display zoom. Magnifying an ultrasound image to create enhanced ultrasound images also suffer from deficiencies. Specifically, a zoomed image portion of an ultrasound image created using interpolation does not have enhanced image quality beyond merely magnification.

The present includes ultrasound imaging techniques and systems for implementing techniques that facilitate independent creation of ultrasound images of a ROI within a subject area. Subsequently, the independently formed ROI ultrasound images can be displayed concurrently with main ultrasound images of the subject area. In being independently formed from the main ultrasound images, the ROI ultrasound images can be enhanced when compared to the main ultrasound images. Enhanced/enhancing, as used herein, can include changing/increasing applicable characteristics of an ultrasound image, e.g. increased spatial resolution, increased contrast resolution, increased temporal resolution, and increased penetration resolution, when compared to the main ultrasound images. For example, and as will be discussed in greater detail later, the ROI ultrasound images can be processed to have increased spatial resolution when compared to the main ultrasound images. This is advantageous over typical magnification techniques that are used to enhance ultrasound images, which only have increased magnification.

Main ultrasound images and ROI ultrasound images generated independently from the main ultrasound images, e.g. enhanced ROI ultrasound images, can be displayed concurrently. This can allow an operator to examine both an overall subject area and a ROI within the subject area simultaneously. This is advantageous over typical acoustic zoom techniques, where the relationship between the ROI ultrasound images and the main ultrasound images is lost at a detriment to ultrasound operators.

Returning back to the example flowchart 200 shown in FIG. 2, at step 202, main channel domain data of a subject area for performing ultrasound imaging of the subject area is collected. Main channel domain data of a subject area can be collected by an applicable ultrasound transducer, such as the ultrasound transducer 110 shown in FIG. 1. Channel domain data, as used herein, includes data generated from transducer elements and from every transmit/receive cycle that is used to produce an ultrasound image. For example, in a 128-channel system that is using a single focus zone and sampling to a depth of 16 cm in a curved array format there might be around 192 transmit receive cycles. Channel domain data can include data that is used to generate an ultrasound image before any processing is done on the data. For example, channel domain data can include data that is generated by an ultrasound transducer before the data is pre-processed for beamforming, before beamforming actually occurs, and/or before the data is post-processed after beamforming to generate an ultrasound image.

At step 204, ROI channel domain data for performing ultrasound imaging of a ROI within the subject area is collected. ROI channel domain data of a ROI within the subject area can be collected by an applicable ultrasound transducer, such as the ultrasound transducer 110 shown in FIG. 1. A ROI in the subject area can include a subset of the subject area. Specifically, a ROI in the subject area can include a subset of the subject area represented in the one or more main ultrasound images.

The ROI channel domain data for the ROI within the subject area can be collected by varying ROI data acquisition parameters used to gather/collect/generate the ROI channel domain data. Specifically, the ROI channel domain data can be collected by varying ROI data acquisition parameters with respect to main channel domain data acquisition parameters used to collect the main channel domain data of the subject area. A data acquisition parameter can include an applicable parameter for collecting channel domain data through an ultrasound transducer. Specifically, data acquisition parameters can include transmit and receive imaging parameters for gathering channel domain data. More specifically data acquisition parameters can include a transmit frequency of ultrasound waves used to gather channel domain data, a transmit waveform design of the ultrasound waves, front-end analog gain, and transmit aperture and focus design. For example, the ROI data can be acquired with a greater ultrasound wave focus than a focus of ultrasound waves used to gather the main ultrasound data. In another example, the ROI data can be collected using ultrasound waves of a first waveform design while the main ultrasound data can be collected using ultrasound waves of a second waveform design that is different from the first waveform design used to gather the ROI data.

At step 206, one or more main ultrasound images of the subject area are formed using the main channel domain data. In particular, ultrasound processing operations can be applied to the main channel domain data to generate the one or more main ultrasound images. Ultrasound processing operations can include applicable operations applied to channel domain data for purposes of generating one or more ultrasound images. Specifically, ultrasound processing operations can include applicable operations applied to channel domain data before post-beamformed data processing/backend processing is applied to generate one or more ultrasound images. More specifically, ultrasound processing operation can include data operations applied to generate beamformed data from channel domain data, which can subsequently be post-processed to form one or more ultrasound images. Additionally, an ultrasound processing operation, as described herein, can include a plurality of sub-operations. Specifically, an ultrasound processing operation can include a plurality of operations that are applied to the channel domain data to process the data according the ultrasound processing operation. For example, an ultrasound processing operation can include both minimum variance operations and phase coherence operations applied to the channel domain data as part of an overall ultrasound processing operation.

Further, an ultrasound processing operation can include an operation for beamforming the channel domain data. Specifically, an ultrasound processing operation can include a beamforming operation for ultimately creating beamformed data from the channel domain data. For example, an ultrasound processing operation can be a coherent beamforming operation, a digital beamforming operation, a synthetic aperture beamforming operation, or an adaptive beamforming operation.

Additionally, an ultrasound processing operation can include backend processing/post-beamformed data processing. Backend processing can include applicable operations for forming ultrasound images from post-beamformed data. For example backend processing can include up sampling, down sampling, log compression, detection, spatial filtering, adaptive filtering, scan conversion, and the like, that facilitate ultrasound image data display.

At step 208, one or more ROI ultrasound images of the ROI are formed from the ROI channel domain data of the ROI within the subject area. Specifically, the one or more ROI ultrasound images of the ROI can be formed independently from the one or more main ultrasound images. More specifically, ultrasound processing operations can be applied to the ROI channel domain data to form the one or more ROI ultrasound images of the ROI independent from the one or more main ultrasound images. In forming the ROI ultrasound images independently from the main ultrasound images, the ROI ultrasound images can be formed by enhancing different aspects of the images beyond simple magnification of the main ultrasound images. For example, the ROI images can have increased spatial resolution, increased contrast resolution, increased temporal resolution, and increased penetration resolution in comparison to the main ultrasound images. Additionally, by forming the ROI ultrasound images independently from the main ultrasound images, as will be discussed in greater detail later, the ROI ultrasound images and the main ultrasound images can be concurrently displayed.

The ROI ultrasound images can be formed independently from the main ultrasound images by controlling varying ROI data acquisition parameters used to gather the ROI channel domain data with respect to the main channel domain data acquisition parameters used to gather the main channel domain data. Specifically, the ROI channel domain data can be gathered independently from the main channel domain data according to ROI channel domain acquisition parameters, and the ROI channel domain data can be used to independently form the ROI ultrasound images. For example, ROI channel domain data can be gathered at a higher ultrasound wave transmission frequency than a frequency of ultrasound waves used to gather the main channel domain data of the subject area. Subsequently, the ROI channel domain data gathered at the higher ultrasound wave transmission frequency can be used to generate the ROI ultrasound images independently from the main ultrasound images.

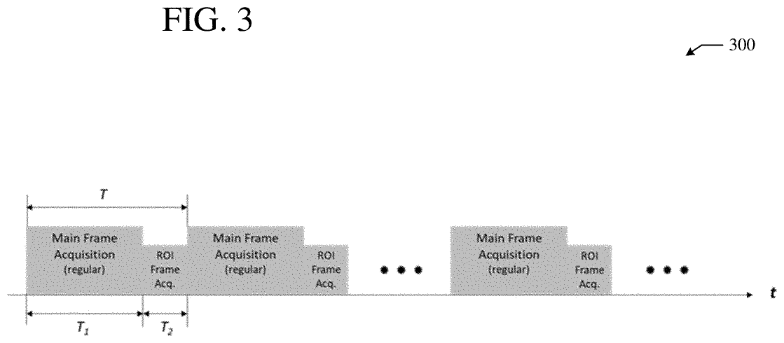

FIG. 3 shows an example imaging sequence 300 of ROI ultrasound frames and main ultrasound frames collected according to varying data acquisition parameters. In particular, the example imaging sequence 300 shown in FIG. 3 can be used to generate the ROI ultrasound images independently from the main ultrasound images. Specifically, both transmit and receive imaging parameters can be selected to have enhanced spatial resolution for the ROI images with minimum impact on the overall frame rate of the main ultrasound images. More specifically, in the example imaging sequence 300, T₂ for ROI frame acquiring is less than/a fraction of T₁ for main frame acquisition. The resultant frame rate is given by Equation 1 below.

1/T=1/(T₁+T₂)<1/T₁. Equation 1

Therefore, the main frame and ROI frame can be 1-to-1 matched with the same frame rate to allow for easier correlation between the ROI ultrasound images and the main ultrasound images. This is advantageous as the ROI frame has a higher TX zone density and a higher RX line density to get stronger focus and better spatial resolution, e.g. corresponding to enhanced ROI ultrasound images.

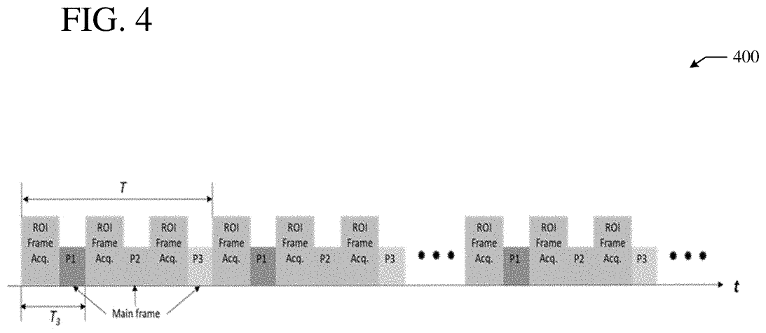

FIG. 4 shows another example imaging sequence 400 of ROI ultrasound frames and main ultrasound frames collected according to varying data acquisition parameters. In the imaging sequence 400 shown in FIG. 4, the frame rate inside the ROI is faster than the frame updates for the main ultrasound images of the subject area. Specifically, the main ultrasound frame rate acquisition for the main ultrasound images can be split into K groups (e.g., P1, P2, P3). The ROI frame time is 1/K of the main frame time, as shown in Equation 2 below.

T₃=T/K (e.g., K=3 in the plot). Equation 2

Accordingly, the ROI frame rate=K*main frame rate. In turn, this can significantly improve the temporal resolution for the image portion inside the ROI when compared to the main ultrasound images.

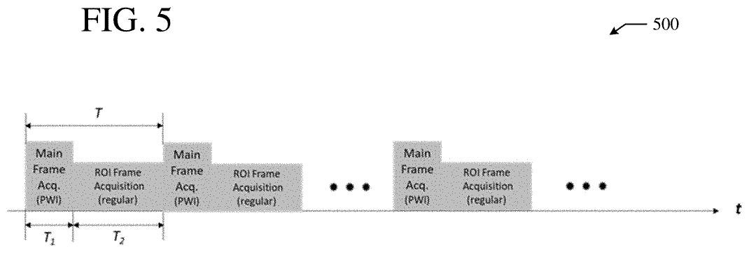

FIG. 5 shows yet another example imaging sequence 500 collected according to varying acquisition parameters. The example imaging sequence 500 shown in FIG. 4 uses plane-wave imaging (PWI) to generate the main frame. As a result T₁ is short when compared to T₂. The resultant frame rate is shown by Equation 3 below.

1/T=1/(T₁+T₂) Equation 3

Specifically, the resultant frame rate can be much faster than a frame rate of conventional full size B-Mode imaging. However, the ROI frame can still use focused TX beams and higher TX zone density to get better focus and/or higher frequency leading to enhanced resolution in the ROI ultrasound images when compared to the main ultrasound images of the subject area. Specifically, both spatial resolution and temporal resolution can be enhanced, when compared to the main ultrasound images, for the ROI ultrasound images.

Returning back to the flowchart 200 shown in FIG. 2, imaging parameters for generating the one or more ROI ultrasound images can be varied to generate the one or more ROI ultrasound images independently from the one or more main ultrasound images. Specifically, imaging parameters for generating the one or more ROI ultrasound images can be varied with respect to imaging parameters used to generate the one or more main ultrasound images, in order to independently form the ROI ultrasound images. Imaging parameters include applicable parameters for controlling application of ultrasound processing operations to generate ultrasound images. Specifically, imaging parameters can indicate specific ultrasound processing operations to apply in order to generate ultrasound images and how to apply the specific ultrasound processing operations to generate the ultrasound images. For example, imaging parameters can specify to apply a specific beamforming operation to generate the ROI images. In another example, imaging parameters can specify values of parameters of a specific beamforming operation to apply in generating the ROI images.

Additionally, backend processing can be varied to generate the one or more ROI ultrasound images independently from the one or more main ultrasound images. Specifically, different backend processing can be applied to generate the ROI ultrasound images than the backend processing applied to generate the main ultrasound images. For example, filtering can be applied to generate the ROI ultrasound images to further generate enhanced ROI ultrasound images when compared to the main ultrasound images.

At step 210, the one or more ROI ultrasound images are displayed concurrently with the one or more main ultrasound images. Specifically, the one or more ROI ultrasound images can be display concurrently with the one or more main ultrasound images in real-time as the images are formed. By concurrently displaying the ROI ultrasound images with the main ultrasound images, a user can concentrate more easily on a reginal image within a given ROI with enhanced image quality and also obtain a visualization of the entire field of view of the subject area. As discussed previously, this is advantageous over current ultrasound imaging techniques, where the relationship between a ROI image and a main ultrasound image is lost through magnification to create the ROI image.

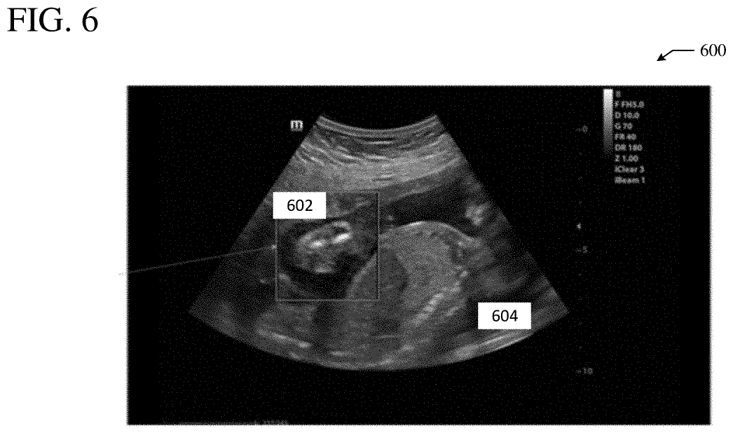

In being displayed concurrently with the main ultrasound images, the ROI ultrasound images can be displayed within the main ultrasound images. Specifically, FIG. 6 shows an example ultrasound image display format 600, where ROI ultrasound images 602, e.g. enhanced ROI ultrasound images, are displayed within main ultrasound images 604. More specifically, the ROI ultrasound images 602 are presented over where the ROI is actually shown in the main ultrasound images 604, e.g. an area in the main ultrasound images 604 corresponding to the ROI. As will be discussed in greater detail later, the ROI ultrasound images 602 themselves can be further magnified while still being displayed in the main ultrasound images 604.

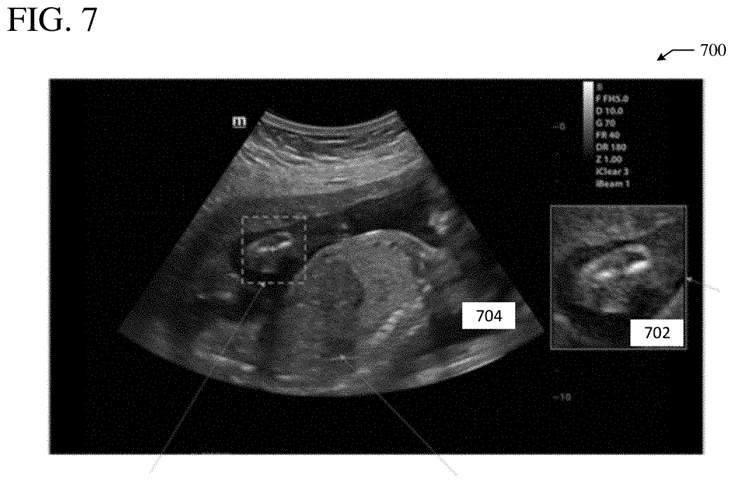

Alternatively, the ROI ultrasound images can be displayed adjacent to the main ultrasound images while being concurrently displayed with the main ultrasound images. FIG. 7 shows an example ultrasound image display format 700, where ROI ultrasound images 702, e.g. enhanced ROI ultrasound images, are displayed adjacent to main ultrasound images 704. As will be discussed in greater detail later, the ROI ultrasound images 702 themselves can be further magnified while still being displayed adjacent to the main ultrasound images 704. The image display format 700 includes an indicator, e.g. dashed line box, showing the ROI in the main ultrasound images 704. The indicator can be movable by an operator. In turn, the ROI ultrasound images 702 can be updated based on movement of the indicator to show a new ROI covered by the indicator. The ROI ultrasound images 702 can be updated based on a user-selected position and shape of the indicator using the techniques described herein.

Returning back to FIG. 2, the various techniques described with respect to the flowchart 200 shown in FIG. 2 can be performed based on input received from an operator/user. Specifically, an operator can provide input, e.g. ROI control input, for controlling enhancement and display of the one or more ROI ultrasound images. ROI control input can specify a ROI, e.g. in a main ultrasound image, to image for forming ROI ultrasound images independently from the main ultrasound image. For example, ROI control input can specify a size and/or a shape of a ROI within the subject area to image for generating one or more ROI ultrasound images. In another example, ROI control input can specify moving the ROI within the subject area, effectively creating a new ROI, in order to generate ultrasound images of the new ROI. In turn, an ultrasound processing system, such as the main processing console 118, can select a ROI based on received ROI control input and subsequently generate ultrasound images for the ROI independently from forming main ultrasound images of a subject area.

ROI control input can specify how to generate ultrasound images of a ROI independently from forming main ultrasound images of a subject area. Specifically, ROI control input can specify one or a combination of data acquisition parameters to use in gathering ROI channel domain data, imaging parameters and corresponding ultrasound processing operations for generating ROI ultrasound images independently from main ultrasound images, and backend processing to apply in generating and displaying the ROI ultrasound images, e.g. displaying the ROI images concurrently with the main ultrasound images. Further, ROI control input can specify different manners to enhance ROI ultrasound images. Specifically, ROI ultrasound images can specify improving one or a combination of spatial resolution, contrast resolution, temporal resolution, and penetration resolution in ROI ultrasound images when compared to main ultrasound images of a subject area. Accordingly a user can control/select what aspects of a ROI to change in generating ROI ultrasound images, e.g. enhanced ROI ultrasound images, independently from main ultrasound images of a subject area. For example, an operator can provide ROI control input specifying to increase temporal resolution in a ROI ultrasound image by a certain amount when compared to a temporal resolution of a main ultrasound image of a subject area.

Additionally, ROI control input can include magnification instructions for magnifying ROI images, e.g. ROI images that are already enhanced according to the techniques described herein. Specifically, magnification instructions can specify a magnification scale factor for magnifying ROI images, e.g. as they are currently displayed with main ultrasound images. Additionally, magnification instructions can specify a magnification region within a ROI to magnify for generating magnified ROI images. In turn, an applicable ultrasound processing system, such as the main processing console 118, can magnify ROI images according to magnification instructions included as part of ROI control input. Subsequently the magnified ROI images can be displayed concurrently with main ultrasound images of a subject area.

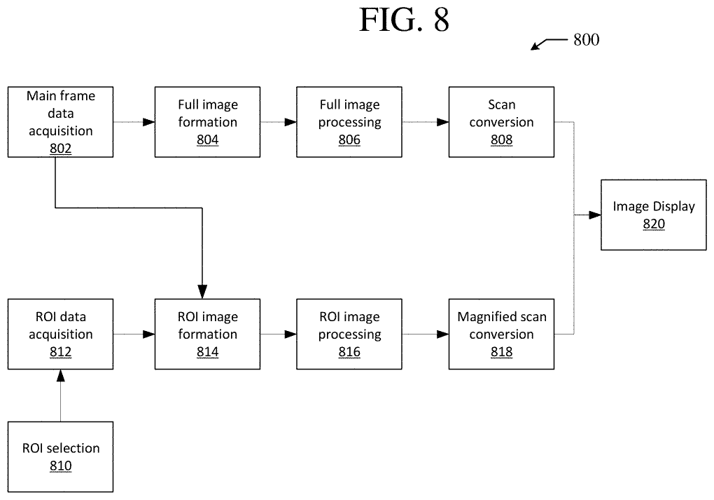

FIG. 8 shows another example flowchart 800 of an example method for independently generating ROI ultrasound images of a subject area from main ultrasound images of the subject area for concurrent display of the ROI and main ultrasound images. At step 802, main frame data of a subject area is acquired. At step 804, the full image/main ultrasound image is formed from the main frame data acquired at step 802. At step 806, the full image is processed, and at step 808 the processed full image is scan converted for display.

Either before or concurrently with the previously described steps, at step 810, a ROI in the subject area is selected. At step 812, ROI data of the selected ROI is acquired. At step 814, the ROI image is formed from the ROI data acquired at step 812. The ROI image can also be formed, at step 814, using the main frame data acquired at step 802. At step 816, the ROI image is processed, and at step 818 the processed ROI image is magnified and scan converted for display. The ROI image can be magnified based on ROI control input received from an operator. Finally, at step 820, both the ROI image and full image are displayed concurrently after scan conversion at steps 808 and 818.

The techniques described herein, including the methods shown in FIGS. 2 and 8, can be applied in an applicable ultrasound imaging mode, such as B-Mode, contrast-enhanced ultrasound (‘CEUS’), CD-Mode, 2D/3D/4D, and the like. Specifically, the techniques described herein are not limited to B-Mode but can also be applied to other modes where improved temporal resolution within a region of interest has substantial clinical benefits, such as CEUS.

This disclosure has been made with reference to various exemplary embodiments including the best mode. However, those skilled in the art will recognize that changes and modifications may be made to the exemplary embodiments without departing from the scope of the present disclosure. For example, various operational steps, as well as components for carrying out operational steps, may be implemented in alternate ways depending upon the particular application or in consideration of any number of cost functions associated with the operation of the system, e.g., one or more of the steps may be deleted, modified, or combined with other steps.

While the principles of this disclosure have been shown in various embodiments, many modifications of structure, arrangements, proportions, elements, materials, and components, which are particularly adapted for a specific environment and operating requirements, may be used without departing from the principles and scope of this disclosure. These and other changes or modifications are intended to be included within the scope of the present disclosure.

The foregoing specification has been described with reference to various embodiments. However, one of ordinary skill in the art will appreciate that various modifications and changes can be made without departing from the scope of the present disclosure. Accordingly, this disclosure is to be regarded in an illustrative rather than a restrictive sense, and all such modifications are intended to be included within the scope thereof. Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, a required, or an essential feature or element. As used herein, the terms “comprises,” “comprising,” and any other variation thereof, are intended to cover a non-exclusive inclusion, such that a process, a method, an article, or an apparatus that comprises a list of elements does not include only those elements but may include other elements not expressly listed or inherent to such process, method, system, article, or apparatus. Also, as used herein, the terms “coupled,” “coupling,” and any other variation thereof are intended to cover a physical connection, an electrical connection, a magnetic connection, an optical connection, a communicative connection, a functional connection, and/or any other connection.

Those having skill in the art will appreciate that many changes may be made to the details of the above-described embodiments without departing from the underlying principles of the invention. The scope of the present invention should, therefore, be determined only by the following claims.

What is claimed is:

1. A method for performing ultrasound imaging comprising:
collecting main channel domain data of a subject area for performing ultrasound imaging of the subject area;
collecting region of interest (“ROI”) channel domain data for performing ultrasound imaging of a ROI within the subject area;
forming one or more main ultrasound images of the subject area using the main channel domain data;
forming one or more ROI ultrasound images of the ROI within the subject area independently from the one or more main ultrasound images of the subject area using the ROI channel domain data of the ROI within the subject area; and
displaying the one or more ROI ultrasound images concurrently with the one or more main ultrasound images.

2. The method of claim 1, wherein the one or more ROI ultrasound images are formed independently from the one or more main ultrasound images by varying ROI data acquisition parameters used in gathering the ROI channel domain data with respect to main channel domain data acquisition parameters used in gathering the main channel domain data.

3. The method of claim 2, wherein the ROI data acquisition parameters include transmit and receive imaging parameters.

4. The method of claim 3, wherein first transmit and receive parameters used to gather the ROI channel domain data are varied with respect to second transmit and receive parameters used to gather the main channel domain data.

5. The method of claim 2, wherein the ROI data acquisition parameters include one or a combination of a transmit frequency of ultrasound waves used to gather the ROI data, a transmit waveform design of the ultrasound waves, front-end analog gain, and transmit aperture and focus design.

6. The method of claim 2, wherein a frame rate of forming the one or more ROI ultrasound images is greater than a frame rate of forming the one or more main ultrasound images.

7. The method of claim 1, wherein the one or more ROI ultrasound images are formed independently from the one or more main ultrasound images by varying ROI imaging parameters used in forming the one or more ROI ultrasound images with respect to main frame imaging parameters used in forming the one or more main ultrasound images.

8. The method of claim 1, wherein in being formed independently from the one or more main ultrasound images, the one or more ROI ultrasound images have one or a combination of an increased spatial resolution, increased contrast resolution, increased temporal resolution, and increased penetration resolution when compared to the one or more main ultrasound images.

9. The method of claim 1, wherein the one or more ROI ultrasound images are formed independently from the one or more main ultrasound images by applying different backend processing to generate the one or more ROI ultrasound images than backend processing applied to generate the one or more main ultrasound images.

10. The method of claim 1, further comprising:
receiving ROI control input from an operator; and
selecting an area within the subject area to serve as the ROI based on the ROI control input received from the operator.

11. The method of claim 10, wherein the ROI control input includes magnification instructions for magnifying the one or more ROI images, the method further comprising:
magnifying the one or more ROI images according to the magnification instructions to generate one or more magnified ROI images; and
displaying the one or more magnified ROI images concurrently with the one or more main ultrasound images.

12. The method of claim 11, wherein the magnification instructions include a magnification scale factor for magnifying the one or more ROI images to generate the one or more magnified ROI images.

13. The method of claim 11, wherein the magnification instructions include a magnification region within the ROI for magnifying within the one or more ROI images to generate the one or more magnified ROI images.

14. The method of claim 10, wherein the ROI control input specifies either or both a shape and a size of the area within the subject area to serve as the ROI.

15. The method of claim 1, wherein the one or more ROI ultrasound images are displayed within the one or more main ultrasound images while displayed concurrently with the one or more main ultrasound images.

16. The method of claim 15, wherein the one or more ROI ultrasound images are displayed within the one or more main ultrasound images in an area in the one or more main ultrasound images corresponding to the ROI.

17. The method of claim 1, wherein the one or more ROI ultrasound images are displayed adjacent to the one or more ultrasound images while displayed concurrently with the one or more main ultrasound images.

18. The method of claim 1, wherein the one or more main ultrasound images and the one or more ROI ultrasound images are generated using B-mode ultrasound imaging, contrast-enhanced ultrasound imaging, CD-mode ultrasound imaging, 2D ultrasound imaging, 3D ultrasound imaging, or 4D ultrasound imaging.

19. A system for performing ultrasound imaging comprising:
an ultrasound transducer configured to:
collect main channel domain data for performing ultrasound imaging of a subject area;
collect region of interest (“ROI”) channel domain data for performing ultrasound imaging of a ROI within the subject area;
a main processing console configured to:
form one or more main ultrasound images of the subject area using the main channel domain data;
form one or more ROI ultrasound images of the ROI within the subject area independently from the one or more main ultrasound images of the subject area using the ROI channel domain data of the ROI within the subject area; and
display the one or more ROI ultrasound images concurrently with the one or more main ultrasound images.

20. A system for performing ultrasound imaging comprising:
one or more processors; and
a computer-readable medium providing instructions accessible to the one or more processors to cause the one or more processors to perform operations comprising:
collecting main channel domain data for performing ultrasound imaging of a subject area;
collecting region of interest (“ROI”) channel domain data for performing ultrasound imaging of a ROI within the subject area;
forming one or more main ultrasound images of the subject area using the main channel domain data;
forming one or more ROI ultrasound images of the ROI within the subject area independently from the one or more main ultrasound images of the subject area using the ROI channel domain data of the ROI within the subject area; and
displaying the one or more ROI ultrasound images concurrently with the one or more main ultrasound images.
